(19) United States
(12) Patent Application Publication
(10) Pub. No.: US 2009/0105747 A1
(43) Pub. Date: Apr. 23, 2009

(54) Vena Cava Filter with Stent

(75) Inventors: Andrzej J. Chanduszko, Chandler, AZ (US); Joshua A. Smale, Chandler, AZ (US)

Correspondence Address: C. R. Bard, Inc.;Bard Peripheral Vascular, Inc., 1415 W. 3rd Street, P.O. Box 1740, Tempe, AZ, 85280-1740, US

(73) Assignee: C.R. Bard, Inc., Murray Hill, NJ (US)

(21) Appl. No.: 12/095,991

(22) PCT Filed: Dec. 1, 2006
(86) PCT No.: PCT/US2006/046146

§ 371 Date: Jul. 31, 2008

Related U.S. Application Data
(60) Provisional application No. 60/748,237, filed on Dec. 7, 2005.

(51) Int. Cl. A61M 29/00; A61F 2/06

(52) U.S. Cl. 606/200; 623/1.15; 623/1.16; 623/1.2

(57) ABSTRACT

An implantable medical device is described, including a filtering element and radially expandable structure. In one variation, the filtering element may include a pluralit of filaments attached to the structure, the filaments being joined together at a proximal en thereof. The filtering element may include strut members and a hub attached to the proxima end of the filaments. The filaments may be made of suture material. In another variation, the filtering element may include a filter with a plurality of legs, the filter being attached to the support structure via a plurality of filaments.

### PRIORITY

This application claims the benefit of priority to U.S. Application No. 60/748,237, filed Dec. 7, 2005, which is incorporated by reference into this application as if fully set forth herein.

### BACKGROUND

Inferior vena cava (IVC) filters are devices configured for insertion into a blood vessel to capture particles that may be present in the blood stream which, if transported to, for example, the lungs could result in serious complications and even death. Typically, IVC filters are utilized in patients who have a contraindication to anticoagulation or in patients developing clinically apparent deep vein thrombosis (DVT) and/or pulmonary embolism (PE). Patients who have recently suffered from trauma, have experienced a heart attack (myocardial infarction), or who have undergone major surgical procedure (e.g., surgical repair of a fractured hip, etc.) may develop clinically apparent DVT. When a thrombus clot loosens from the site of formation and travels to the lung, it may cause PE, a life-threatening condition. An IVC filter may be placed in the circulatory system to intercept one or more clots and prevent them from entering the lungs. IVC filters are either permanent or retrievable.

There are many different configurations for IVC filters, including those that include a central hub from which extend a plurality of struts that form filter baskets having a conical configuration, such as disclosed in U.S. Pat. No. 6,258,026, which is incorporated by reference into this application as if fully set forth herein. Other IVC filter configurations utilize wires and/or frame members to form straining devices that permit flow of blood while trapping larger particles. IVC filters are generally configured for compression into a small size to facilitate delivery into the inferior vena cava and subsequent expansion into contact with the inner wall thereof. The IVC filter may later be retrieved from the deployed site by compressing the legs, frame members, etc., depending on the filter configuration. Typically, an IVC filter will include hooks or anchoring members for anchoring the filter in position within the inferior vena cava. The hooks may be more elastic than the legs or frame members to permit the hooks to straighten in response to withdrawal forces, which facilitate withdrawal from the endothelium layer of the blood vessel without risk of significant injury to the vessel wall.

Intraluminal prostheses used to maintain, open, or dilate blood vessels are commonly known as stents. Stents are either self-expanding or balloon expandable. Self-expanding stents are delivered to a blood vessel in a collapsed condition and expand in vivo following the removal of a constraining force and/or in the presence of an elevated temperature (due to material properties thereof), whereas balloon expandable stents are generally crimped onto a balloon catheter for delivery and require the outwardly directed force of a balloon for expansion.

Related disclosure of a stent and filter unit are shown and described in U.S. Pat. No. 4,655,771 and U.S. Pat. No. 6,712,834, which are incorporated by reference into this application as if fully set forth herein. However, these stent-filter units are believed not to be retrievable after implantation into a blood vessel. The following references relate to blood filters: U.S. Pat. No. 4,990,156; U.S. Pat. No. 5,375,612; U.S. Pat. No. 5,634,942; U.S. Pat. No. 5,709,704; U.S. Pat. No. 5,853,420; U.S. Pat. No. 6,013,093; U.S. Pat. No. 6,214,025; U.S. Pat. No. 6,241,746; U.S. Pat. No. 6,245,012; U.S. Pat. No. 6,436,121; U.S. Pat. No. 6,506,205; US Publication No. 2003/0097145; US Publication No. 2003/0176888; and US Publication No. 2004/0073252, which are incorporated by reference in their entireties into this application.

In certain circumstances, applicants have recognized that it would be desirable to combine the filtering function of an IVC filter and one or more advantageous functions of a stent in a blood vessel and to provide for the ability to remove the filter after the threat of emboli or blood clots has been reduced. Thus, described herein are embodiments of an implantable medical device that includes an IVC filter and a stent.

### BRIEF SUMMARY OF THE INVENTION

Accordingly, implantable medical devices including one or more filters and a stent are described herein. In one embodiment, an implantable medical device includes a radially expandable structure, having an open proximal end and an open distal end, and a plurality of filaments attached to the structure proximate at least one of the ends, the filaments being connected together to define a first filtering element. In another embodiment, an implantable medical device includes a filter including a plurality of legs joined at a proximal end to a hub, a radially expandable structure, having an open proximal end and an open distal end, and a plurality of filaments attaching the filter to the structure. In yet another embodiment, an implantable medical device includes a radially expandable structure, having an open proximal end and an open distal end defining a longitudinal axis extending therethrough, and a filter including a plurality of appendages disposed partly inside the radially expandable structure and joined at a proximal end to a hub.

In another embodiment, a method of filtering blood in a blood vessel includes introducing an implantable medical device into a blood vessel in a collapsed configuration, deploying the implantable medical device into the blood vessel, the device translating to an expanded configuration having a support structure for the blood vessel wall and a filter structure for blood flowing through the vessel, and separating the filter structure from the support structure after a predetermined time period.

These and other embodiments, features and advantages will become more apparent to those skilled in the art when taken with reference to the following more detailed description of the invention in conjunction with the accompanying drawings that are first briefly described.

### BRIEF DESCRIPTION OF THE DRAWINGS

FIG. 1 is a side view of one embodiment of an implantable medical device including a filter and a stent.

FIG. 2 is a side view of another embodiment of an implantable medical device, including a filtering element and a stent.

FIG. 3 is a side view with a partial cut-away portion of another embodiment of an implantable medical device, including a first and second filtering element and a stent.

FIG. 4 is a side view with a partial cut-away portion of another embodiment of an implantable medical device including a filter and a stent.

FIG. 5 is a side view of one embodiment of a filter with a centralized hub.

### DETAILED DESCRIPTION OF THE PREFERRED EMBODIMENTS

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

While the examples provided herein are discussed with respect to IVC filters, it should be appreciated that the filter embodiments described herein could be used for filter applications that do not involve placing a filter device in the inferior vena cava. In other words, the filters described herein are not limited to IVC applications. Moreover, as used herein, the term “suture material” means a material that is, or could be, used as a suture thread by a surgeon, including, for example, synthetic polymers, polyglycolic acid (PGA), polylactic acid (PLA), polydioxanone (PDS), polyglactin, nylon, polypropylene (prolene), silk, catgut, non-absorbable/non-biodegradable materials, and combinations thereof. Included in this term are both monofilament and multifilament suture materials. Further, as used herein the term “bio-resorbable” includes a suitable biocompatible material, mixture of various biocompatible materials or partial components of biocompatible material being altered into other materials by an agent present in the environment (e.g., a biodegradable material that degrades via a suitable mechanism such as hydrolysis when placed in biological tissue); such materials being removed by cellular activity or incorporated into the cellular structure (i.e., bioresorption, bioresorping, bioabsorption, or bioresorbable), such materials being degraded by bulk or surface degradation (i.e., bioerosion such as, for example, a water insoluble polymer that turns water-soluble in contact with biological tissue or fluid), or such materials being altered by a combination of one or more of biodegradable, bioerodable or bioresorbable activity when placed in contact with biological tissue or fluid.

Also, as used herein, the term “hook” means a member configured to engage a blood vessel wall, examples of which are provided in U.S. Pat. No. 6,258,026, which is incorporated by reference as if fully set forth herein. The term “stent” as used herein means any radially expandable structure, having an open proximal end and an open distal end, configured for insertion into a blood vessel and includes both self-expanding and balloon expandable types. Possible materials for the stent and filter described herein include a suitable biocompatible material such as, for example, stainless steel, noble metals and their alloys, shape memory metals, shape memory alloys, super elastic metal, super elastic shape memory metal alloys, linear elastic shape memory metal, metal alloys, shape memory polymers, polymers, bio-resorbable materials (e.g., metal alloys such as those shown and described in U.S. Pat. No. 6,287,332; and U.S. Patent Application Publication No. 2002/0004060, which are incorporated by reference in their entireties into this application), and combinations thereof.

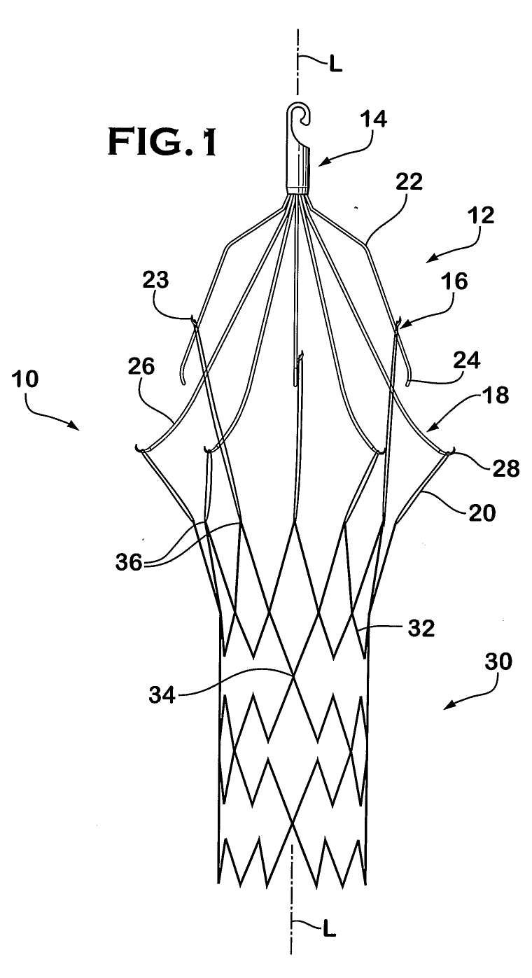

Referring now to FIG. 1, one embodiment of an implantable medical device including a filter and a stent is illustrated. Implantable medical device 10 includes a filter 12 and a stent 30 that are connected by filaments 20. In one embodiment, the filaments are made of suture material, although in other embodiments, the filaments are made of a bio-resorbable material or any of the materials discussed above with respect to possible materials for the stent and filter. The filter 12 and the stent 30 are illustrated in an expanded configuration, defining an expanded perimeter of the implantable medical device 10. For delivery of the device 10 to a blood vessel, the filter 12 and stent 30 are compressed to a collapsed configuration, defining a collapsed perimeter of the device 10 smaller than the expanded perimeter of the device 10. For actual delivery, the device 10 can be self-expanding due its intrinsic characteristic or via a separate expansion agent (e.g., balloon expansion).

In the embodiment shown in FIG. 1, the filter 12 includes a plurality of arms 16 attached at a proximal end thereof to a hub 14 and a plurality of legs 18 also attached at a proximal end thereof to the hub 14. A similar configuration for a filter is disclosed in U.S. Pat. No. 6,258,026. The hub 14 is shown having a configuration of a retrieval member with a hook-like design, although in other embodiments, the hub 14 forms a sleeve as known to one skilled in the art. The arms 16 and legs 18 may be attached together or to each other as well as to the hub 14. The arms 16 in this embodiment are shorter in length than the legs 18 and extend first outwardly with respect to a longitudinal axis L of the implantable medical device 10 to a shoulder 22 and then distally with respect to the hub 14 and angularly with respect to the shoulder 22. The arms may provide a centering function to the filter 12 and, although shown in this embodiment without hooks or vessel-engaging members on their distal ends, may include hooks in other embodiments. The legs 18 of the filter 12 extend angularly with respect to the longitudinal axis L of the implantable medical device 10 and include a junction 26 near a distal end thereof at which point the legs 18 diverge at a greater angle from the longitudinal axis L, terminating in a hook 28. In other embodiments, less than all the legs 18 may terminate in a hook 28. Details of the hooks are shown and described in U.S. patent application Ser. No. 11/429,975, filed May 9, 2006, which application is incorporated by reference in its entirety into this application.

The hook 28 can be configured for engaging the wall of the blood vessel into which the filter 12 can be deployed and may be made of the same material as the filter 12, or a different material, examples of which are provided above with respect to possible materials for the filter and stent. The hook 28 may be formed with the leg 18 during manufacture, thus being integral therewith, or may be attached subsequent to formation of each by any attachment method known to one skilled in the art (e.g., welding, adhesive bonding, solvent bonding, etc.). In one embodiment, the hook 28 contains a linear portion connected to an arcuate portion that terminates in a point, as shown and described in U.S. Pat. No. 6,258,026. In one embodiment, the arcuate member has a cross-sectional area smaller than the cross-sectional area of the linear portion, as shown and described in U.S. Pat. No. 6,258,026.

Both the arms 16 and legs 18 may be circumferentially spaced equidistant from one another or, alternatively, may be arranged in an unbalanced configuration. The lengths of the arms 16 and legs 18 may be approximately the same as one another or may have different lengths, although generally the arms 16 will have a shorter length than the legs 18. The number of arms 16 and legs 18 can be wide-ranging (e.g., 2, 3, 4, 6, 12, etc.), but in a preferred embodiment, the filter 12 contains six arms 16 and six legs 18. As mentioned, one or more of the arms 16 and one or more of the legs 18 may include a hook 28 at a distal end thereof. A hook may also be positioned along the length of one or more of the arms 16, such as hook 23, and/or one or more of the legs 18 to provide an engaging member for engaging the wall of a blood vessel and/or as an attachment location for the filament 20.

The stent 30, as discussed above, can be any radially expandable structure as known to one skilled in the art, such as the stents shown and described in U.S. Pat. No. 5,707,386, U.S. Pat. No. 5,716,393, U.S. Pat. No. 5,860,999, U.S. Pat. No. 6,053,941, and U.S. Pat. No. 6,572,647, which are incorporated by reference in their entirety into this application. As illustrated, the stent 30 includes struts 32 and connecting segments 34. At both ends of the stent 30, the struts converge to provide a plurality of peaks 36. A substantial portion of the stent, including a majority of an outside surface and/or a majority of an inside surface may be covered by a bio-compatible polymer, such as, for example, Dacron, polyester, PTFE, ePTFE, polyurethane, polyurethane-urea, siloxane, and combinations thereof. Materials for stent coverings, configurations of stent/covering combinations, and different methods for combining stents and coverings are disclosed, for example, in U.S. Pat. No. 5,749,880, U.S. Pat. No. 6,124,523, U.S. Pat. No. 6,398,803, U.S. Pat. No. 6,451,047, U.S. Pat. No. 6,558,414, U.S. Pat. No. 6,579,314 and U.S. Pat. No. 6,620,190, which are incorporated by reference in their entirety into this application.

Filaments 20 connect stent 30 to the filter 12, the filaments 20 being attached to one or more arms 16 and/or one or more legs 18 of the filter 12 at an attachment location thereon (e.g., hooks 23, 28) and to peaks 36 of the stent 30, or other attachment locations along the body of the stent 30. In the embodiment of FIG. 1, the filaments 20 are attached to the arms 16 and the legs 18 of the filter 12 and the peaks 36 of the stent 30. The filaments 20 may be attached to the filter 12 and the stent 30 by wrapping the filament 20 one or more times around an attachment location on the filter 12 and stent 30, tying the filament 20 to an attachment location on the filter 12 and the stent 30, heating the filament 20 adjacent to an attachment location on the filter 12 and the stent 30 to create a bond therebetween, applying an adhesive to the filament 20 and/or an attachment location on the filter 12 and the stent 30, applying a solvent to the filament 20 and/or an attachment location on the filter 12 and the stent 30, etc. Of course, other possibilities for attaching the filament 20 to an attachment location on the filter 12 and the stent 30 known to one skilled in the art are also within the scope of this invention.

In yet another embodiment, the filter 12 may be attached to stent 30 by coupling the filter hooks 28 to a portion of the structure of the stent (e.g., between peaks or valleys of the stent struts). In such embodiment, the hooks 28 would still be able to be deformed toward a more straightened profile, which would allow the filter 12 to be retrieved from the blood vessel.

By virtue of the filament 20, which can be resorbed by the mammalian body, the filter 12 can be recovered separately from the stent. For example, where the stent-filter 10 is utilized as a distal embolic protection device, the filter 12 can be removed once the clinician is confident that no emboli would be dislodged by the implantation of the stent or by the expansion of the stent via balloon angioplasty.

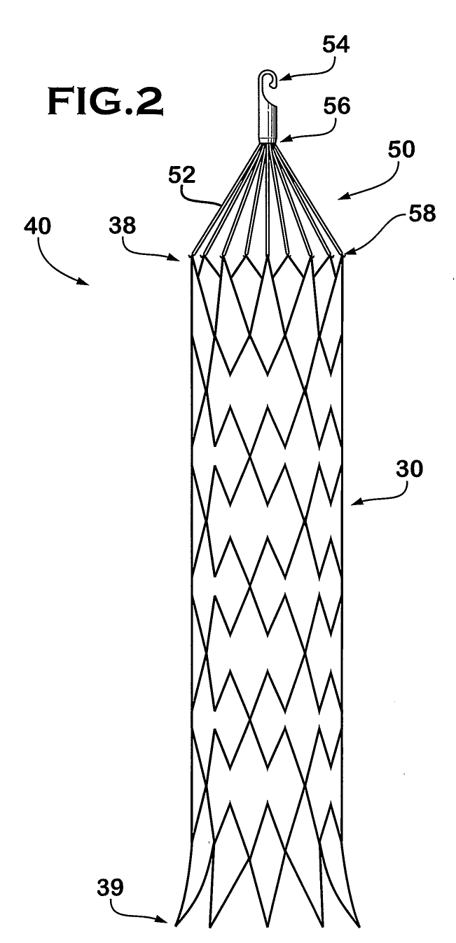

FIG. 2 illustrates another embodiment of an implantable medical device including a filter and a stent. Implantable medical device 40 includes a filtering element 50 and a stent 30. The stent 30 is as described above and may include a bio-compatible covering. Filtering element 50 includes a plurality of filaments 52 that are joined together at a proximal end 56 and attached to the proximal end 38 of the stent 30 at a distal end 58. Attached to the proximal end 56 of the filaments 52 is a hub 54, which has the configuration of a retrieval member with a hook-like design, although in other embodiments, the hub 54 forms a sleeve as known to one skilled in the art. The filaments 52 in a preferred embodiment are made of suture material, but could also be made of a bio-resorbable material or any of the materials discussed above with respect to possible materials for the filter and the stent. The filaments 52 may be attached to the stent 30 by any method described above in connection with FIG. 1 or the filaments 52 can be attached directly from the filter to the stent or sleeve.

By virtue of the filaments, shown in FIG. 2, the filter 50 and stent 30 can be implanted without regard for the direction of blood flow due the utilization of the filament 52. Where blood flow is from one end of the stent toward the filter, as shown in FIG. 2, the filaments 52 allow the filter to extend outside of the stent 30. Where blood flow is in the opposite direction, the filaments 52 allow the filter 50 to achieve its intended filtering function by moving inside the stent 30 (not shown) in the direction of blood flow. This design feature is believed to be advantageous in that one delivery device can be used to deliver the stent and filter from the femoral vein or jugular artery.

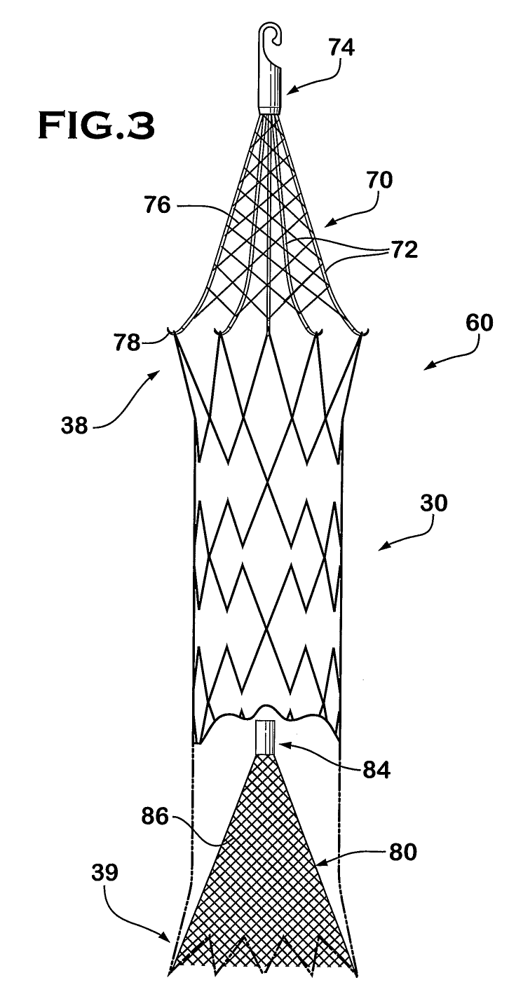

In yet another embodiment, a second filtering element similar to filtering element 50 can be connected to the distal end 39 of the stent, such as illustrated in FIG. 3. The second filtering element 50 can be delivered without regard to the direction of blood flow, as in the embodiment shown in FIG. 2, via a single delivery device from one of the jugular artery or femoral vein.

FIG. 3 illustrates another embodiment of an implantable medical device including a filter and a stent. In the embodiment of FIG. 3, implantable medical device 60 includes a stent 30, a first filter 70, and a second filter 80. The stent 30 is as described above and may include a bio-compatible covering. The first filter 70 includes strut members 72 that are joined together at a proximal end thereof and attached to a hub 74, which has the configuration of a retrieval member with a hook-like design, although in other embodiments, the hub 54 forms a sleeve as known to one skilled in the art. The strut members 72 in a preferred embodiment are made of a bio-resorbable material, but may also be made of any of the materials discussed above with respect to the filter and the stent. Attached to a distal end of the strut members 72 are hooks 78 in the embodiment of FIG. 3, although in other embodiments, some or all of the strut members 72 do not have hooks attached to their distal ends. The hooks 78 (or distal ends of the strut members 72) are directly attached to the stent at a proximal end 38 of the stent (e.g., to the peaks 36). A plurality of filaments 76 can be attached to the strut members 72 in such a way as to form a mesh-like structure. One or more filaments 76 may also be attached to the stent 30, either at a proximal end of the stent or along the length of the stent 30. The filaments 76 in a preferred embodiment are made of suture material, but could also be made of a bio-resorbable material or any of the materials discussed above with respect to possible materials for the filter and the stent. The filaments 76 may be attached to the strut members 72 and the stent 30 by any method described above in connection with the attachment of the filaments 20 to the filter 12 and stent 30 in FIG. 1.

Shown in the cut-away portion of the stent 30 at the distal end 39 is a second filter 80. The second filter 80 can be configured similar to filter 70 including strut members, a hub, filaments and hooks. The distal end of the second filter 80 and/or the hooks can be attached directly to the distal end 39 of the stent 30 (e.g., at peaks 36). As with the first filter 70, the filaments 86 can be attached to the strut members, forming a mesh-like structure, and can also be attached to points along the distal end 39 of the stent 30. The filaments 86 in a preferred embodiment are made of suture material, but could also be made of a bio-resorbable material or any of the materials discussed above with respect to possible materials for the filter and the stent. The filaments 86 may be attached to the strut members and the stent 30 by any method described above in connection with the attachment of the filaments 20 to the filter 12 and stent 30 in FIG. 1. In the embodiment shown in FIG. 3, the second filter 80 does not include struts, the filaments 86 being attached directly to the hub 84 and to the distal end 39 of the stent 30. With no struts, the filter has an increased range of motion allowing it to move in any direction, depending on the direction of blood flow. The hub 84 is shown with the configuration of a sleeve, although in other embodiments, the hub may include a retrieval member similar to that of hub 74. As with the embodiments shown and described in FIG. 2, the filters 70 and 80 can be formed from a flexible material or from a filament material so that each filter forms a generally conical shape that converges toward a longitudinal axis of blood flow, i.e., a generally conical shape regardless of the direction of blood flow to provide for the advantages previously described in relation to FIG. 2.

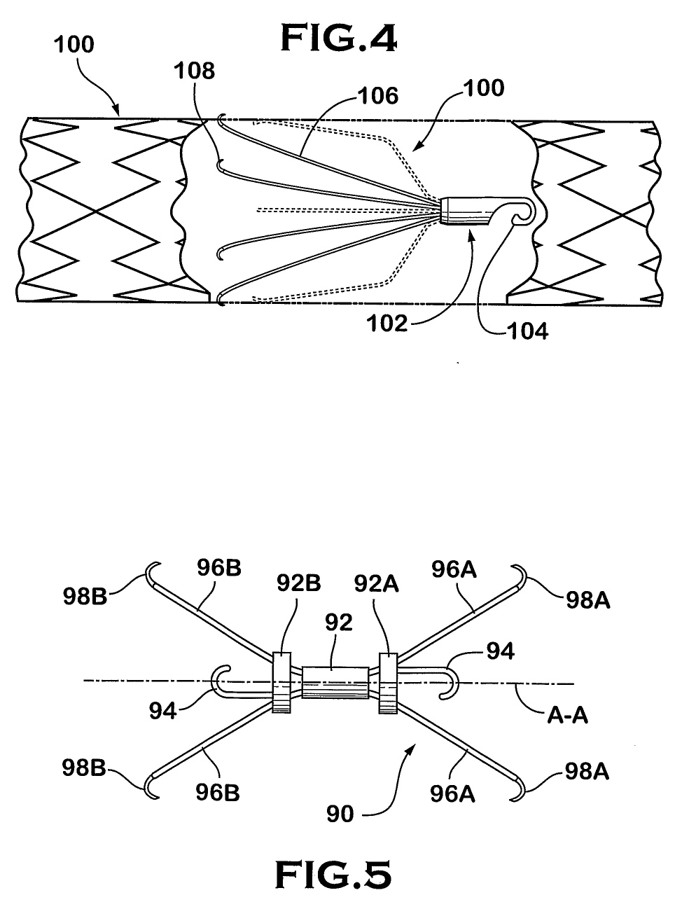

Alternatively, as shown in FIG. 4, a filter 100 may be coupled to a bio-resorbable stent 110, in which after a suitable time period subsequent to implantation, the stent 110 is resorbed into the vessel wall while leaving the filter in place to filter blood for emboli or clots. In this embodiment, the filter 100 may have a single conic structure defined by appendages 106 with a generally centralized hub 102 which can include a snareable hook 104. Appendages 106 can be coupled to anchoring hooks 108 (which are similar to previously described hooks 28). Alternatively, for greater level of filtration, two conical structures can be coupled to each other via a single hub or an intermediate connector between two hubs (see FIG. 5). The conical structures may include appendages that extend in the same direction or in opposite directions. In one of the many preferred embodiments, as mentioned, the filter 100 may have the configuration shown in FIG. 5.

In FIG. 5, the generally centralized hub 92 includes two members 92A and 92B that are slidable with respect to hub 92. The slidable members 92A and 92B allow a recovery device to engage at least one of the members 92A and 92B and slide the member(s) relative to the hub 92. For example, as the slidable member 92A moves to the right relative to hub 92 in FIG. 5, appendages 96A are compressed from generally conical configuration toward a generally cylindrical configuration, thereby separating the hooks 98A from the blood vessel wall (not shown). Subsequently, the appendages 96A and hooks 98A are retracted into a lumen of a recovery catheter. To continue recovery of the filter 90, the recovery device (e.g., a cone type retrieval device shown and described in U.S. Pat. No. 6,156,055) engages the appendages 96B proximate the slidable member 92B to continue pulling the filter 90 toward the right of FIG. 5. This retraction of the filter 90 forces the hooks 98B to distort toward a straightened configuration, allowing for separation of the hooks 98B from the blood vessel wall. Continued movement of the filter 90 in the same direction allows for retraction of the appendages 96B and hooks 98B into the recovery catheter of the recovery device.

In the preferred embodiments of FIGS. 1-5, the filter has a diameter ranging from about 4 millimeters to about 60 millimeters, preferably about 40 millimeters and an overall length ranging from about 10 millimeters to about 100 millimeters, preferably about 40 millimeters; the appendages are formed from a circular cross-section Nitinol wire (although the wire can be cut from a hollow metal tube), having a first cross sectional area, with hooks having a second cross-sectional area less than the first cross sectional area and preferably about 50% to 80% of the first cross-sectional area. Details of the hooks 28 and retrieval member for one embodiment in the range of various sizes of filters are provided in U.S. patent application Ser. No. 11/429,975, filed May 9, 2006, which application is incorporated by reference in its entirety into this application. Retrieval of the preferred filter embodiments shown and described herein can be accomplished via the use of a snare-like filament or via a cone type retrieval device shown and described in U.S. Pat. No. 6,156,055, which is incorporated by reference in its entirety into this application.

Where the filter or stent is to be utilized with bio-active agents to control the formation of emboli, bio-active agents can be coated to a portion or the entirety of the filter for controlled release of the agents once the filter is implanted. The bio-active agents can include, but are not limited to, vasodilator, anti-coagulants, such as, for example, warfarin and heparin.

Other bio-active agents can also include, but are not limited to agents such as, for example, anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II_{b}/IIIₐ inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

Additionally, where it is desired to separate the filter from the stent without waiting for bio-resorption of the bio-resorbable filament, a suitable material can be utilized with the filament where the material changes chemical structure upon exposure to a predetermined wavelength of radiation (e.g., UV or visible light). In one embodiment, the bio-resorbable filament can be provided with a water repellant coating that prevents body fluids from degrading the resorbable material. Once exposed to the predetermined wavelength of radiation, the water repellant coating dissolves or becomes porous so that hydrolytic or enzymatic degradation of the underlying resorbable material can begin. In another example, exposure to a specific wavelength of light causes the light-activated material to change structure to thereby allow separation between the filter and stent for recovery of the filter. In one example, the light can be UV light, visible light or near infrared laser light at a suitable wavelength (e.g., 800 nanometers) to which tissues are substantially transparent to such wavelength and the coating material can be preferably polyethylene with a melting point of about 60 degrees Celsius mixed with biocompatible dyes that absorb light in the such wavelength (e.g., indocyanine green, which is a dye which can absorbs around 800 nm and is biocompatible). The biocompatible dye absorbs the light energy, thereby raising the temperature in the polymer to about 60 degrees Celsius or higher. Upon attainment of the melting point temperature, e.g., 60 degrees Celsius, the polymer structurally weakens thereby allowing the separation of components of the filter or the filter to the stent.

It should be noted that not only can the stent structure be bio-resorbable, various combinations of the bio-resorbable and non-bioresorbable stent and filter can be utilized. For example, the stent (or selected portions of the stent) can be non-bio-resorbable while the filter (or selected portion of the filter) is also bio-resorbable, the stent (or selected portions) can be bio-resorbable whereas the filter is not, or both the stent and filter (or selected portions of the stent and filter) are not bio-resorbable. Moreover, while anchoring hooks have been shown and described in relation to the filter, such hooks can also be utilized with the stent to prevent migration of the stent.

This invention has been described and specific examples of the invention have been portrayed. While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

What is claimed is:

1. An implantable medical device, comprising:
a radially expandable structure, having an open proximal end and an open distal end; and
a plurality of filaments attached to the structure proximate at least one of the ends, the filaments being connected together to define a first filtering element.

2. The implantable medical device according to claim 1, wherein a retrieval member is attached to the filtering element.

3. The implantable medical device according to claim 1, wherein the filtering element comprises strut members having a distal end attached to the structure.

4. The implantable medical device according to claim 3, wherein the strut members have a proximal end attached to a retrieval member.

5. The implantable medical device according to claim 1, further comprising a second filtering element attached to the structure proximate the other of the ends, the second filtering element including a plurality of filaments connected together.

6. The implantable medical device according to claim 1, wherein the structure comprises structure selected from the group consisting of a self-expanding stent, balloon-expandable stent, a stent having a substantial portion of the stent covered in a bio-compatible polymer, and combinations thereof.

7. The implantable medical device according to claim 6, wherein the bio-compatible polymer is selected from a group consisting essentially of Dacron, polyester, PTFE, ePTFE, polyurethane, polyurethane-urea, siloxane, and combinations thereof.

8. The implantable medical device according to claim 7, further comprising a framework having a plurality of first ends connected to the structure and a plurality of second ends connected to each other, the framework comprising a bio-resorbable material.

9. The implantable medical device according to claim 1, wherein the filaments comprise a resorbable material.

10. The implantable medical device according to claim 1, wherein the filaments comprise suture material.

11. The implantable medical device according to claim 1, wherein each of the filaments is connected to an anchoring device having a curved profile.

12. An implantable medical device, comprising:
a filter including a plurality of legs joined at a proximal end to a hub;
a radially expandable structure, having an open proximal end and an open distal end; and
a plurality of filaments attaching the filter to the structure.

13. The implantable medical device according to claim 12, wherein the filter comprises a plurality of arms having a length less than a length of the legs, the arms joined at a proximal end to the hub.

14. The implantable medical device according to claim 13, wherein the filaments are attached to one or more of at least one of the arms and the legs at an attachment location.

15. The implantable medical device according to claim 14, wherein a distal end of the arms comprise a curved portion.

16. The implantable medical device according to claim 12, wherein a distal end of at least one of the legs terminates in a hook.

17. The implantable medical device of claim 16, wherein the hook comprises a generally curved profile having a cross-sectional area smaller than a cross-sectional area of at least the leg.

18. The implantable medical device according to claim 12, wherein the hub comprises a retrieval member.

19. The implantable medical device according to claim 12, wherein the structure comprises structure selected from a group consisting of a self-expanding stent, balloon-expandable stent, a stent having a substantial portion of the stent covered in a bio-compatible polymer, and combinations thereof.

20. The implantable medical device according to claim 19, wherein the bio-compatible polymer is selected from a group consisting essentially of Dacron, polyester, PTFE, ePTFE, polyurethane, polyurethane-urea, siloxane, and combinations thereof.

21. The implantable medical device according to claim 12, wherein at least one of the filter and radially expandable structure comprises a bio-resorbable material.

22. The implantable medical device according to claim 12, wherein the filaments comprise suture material.

23. The implantable medical device according to claim 12, wherein at least a portion of the filter is in contact with the structure.

24. An implantable medical device, comprising:
a radially expandable structure, having an open proximal end and an open distal end defining a longitudinal axis extending therethrough; and
a filter including a plurality of appendages disposed partly inside the radially expandable structure and joined at a proximal end to a hub.

25. The device of claim 24, wherein the plurality of appendages comprises first appendages that extend obliquely with respect to the longitudinal axis in a first direction.

26. The device of claim 25, wherein the plurality of appendages comprises second appendages that extend obliquely with respect to the longitudinal axis in at least one of a first direction and second direction opposite to the first direction.

27. The device of claim 26, wherein at least one of the appendages terminates in a hook.

28. The device of claim 27, wherein the hook comprises a generally curved profile having a cross-sectional area smaller than a cross-sectional area of one of the plurality of appendages.

29. The device of claim 28, wherein the hub further comprises a member that translates with respect to the hub along the longitudinal axis so as to compress the appendages in a direction generally parallel to the longitudinal axis.

30. A method of filtering blood in a blood vessel, comprising:
introducing an implantable medical device into a blood vessel in a collapsed configuration;
deploying the implantable medical device into the blood vessel, the device translating to an expanded configuration having a support structure for the blood vessel wall and a filter structure for blood flowing through the vessel; and
separating the filter structure from the support structure after a predetermined time period.

31. The method of claim 30, wherein the separating further comprises removing the filter from the blood vessel.

32. The method of claim 30, wherein the separating further comprises bioresorbing the support structure in the blood vessel.
